(19) United States
(12) Patent Application Publication
(10) Pub. No.: US 2021/0085696 A1
(43) Pub. Date: Mar. 25, 2021

(54) FORMULATIONS COMPRISING CHELATORS, PERMEATION ENHANCERS AND HYDROXYETHYL CELLULOSE FOR TREATING OPHTHALMIC DISORDERS

(71) Applicant: LIVIONEX, Inc., Los Gatos, CA (US)

(72) Inventors: Rajiv BHUSHAN, Mountain View, CA (US); Jerry GIN, Sunnyvale, CA (US); Amit GOSWAMY, Los Gatos, CA (US)

(21) Appl. No.: 16/967,634

(22) PCT Filed: Mar. 13, 2019
(86) PCT No.: PCT/US2019/022077

§ 371 Date: Aug. 5, 2020

Related U.S. Application Data
(60) Provisional application No. 62/626,541, filed on Feb. 5, 2018.

(51) Int. Cl. A61K 31/65; A61K 31/4706; A61K 47/20; A61K 47/38; A61K 47/32; A61K 47/18

(52) U.S. Cl. CPC A61K 31/65; A61K 31/4706; A61K 47/183; A61K 47/38; A61K 47/32; A61K 47/20

(57) ABSTRACT

An ophthalmic formulation, comprising a chelator (such as EDTA and its salts), and a transport enhancer (such as Methyl Sulfonyl Methane; MSM) and an effective amount of a viscoelastic polymer (such as hydroxymethyl cellulose; HEC) is provided. Together, the combination of the two substances unexpectedly and beneficially reduces discomfort associated with and increases efficacy of chelator/transport enhancer as compared to formulations without the viscoelastic polymer.

### CROSS-REFERENCE TO RELATED APPLICATIONS

This application is a 371 National Stage filing of international application filed under the Paris Convention Treaty (PCT) No. PCT/US2019/022077 filed Mar. 13, 2019 which claims priority to U.S. Provisional Patent Application Ser. No. 62/626,541 filed Feb. 5, 2018 and titled FORMULATIONS COMPRISING CHELATORS, PERMEATION ENHANCERS AND HYDROXYETHYL CELLULOSE FOR TREATING OPHTHALMIC DISORDERS, the contents of which are incorporated herein in their entirety.

### TECHNICAL FIELD OF THE INVENTION

This invention relates generally to the field of treatment of ophthalmic disorders, including the adverse ocular conditions often associated with aging. More particularly, the invention pertains to the treatment of conditions associated with the presence of macromolecular aggregates such as may be present in the eye. In particular, the invention relates to antimicrobial compositions containing a transport enhancer, a chelating agent and hydroxyethylcellulose (HEC). In one exemplary embodiment, it relates to such compositions which contain MSM, chelators and HEC.

### BACKGROUND OF THE INVENTION

Progressive, age-related changes of the eye, including normal as well as pathological changes are an inevitable part of life in humans and other mammals. Many of these changes seriously affect both the function and the cosmetic appearance of the eyes. These changes include: development of cataracts; hardening, opacification, reduction of pliability, and yellowing of the lens; yellowing and opacification of the cornea; presbyopia; clogging of the trabeculum, leading to intraocular pressure build-up and glaucoma; increased floaters in the vitreous humor; stiffening and reduction of the dilation range of the iris; age-related macular degeneration (AMD); formation of atherosclerotic deposits in retinal arteries; dry eye syndrome; and decreased sensitivity and light level adaptation ability of the rods and cones of the retina. Age-related vision deterioration includes loss in visual acuity, visual contrast, color and depth perception, lens accommodation, light sensitivity, and dark adaptation. Age-related changes also include changes in the color appearance of the iris, and formation of arcus senilis.

The inventors of the present application have previously disclosed formulations comprising chelators and permeation enhancers for treatment of ophthalmic disorders. International application no. PCT/US2006/027686 (WO2007011875) disclosed formulations for reducing macromolecular aggregates in the eye with formulations comprising metal chelators and charge-masking agents such as MSM and EDTA. US Patent application no. 20060177430 disclosed formulations containing a biocompatible chelating agent, an effective permeation enhancing amount of an ophthalmic permeation enhancer such as methylsulfonylmethane (MSM), an anti-AGE (advanced glycation endproducts) agent for treating adverse ocular conditions.

However, it has been observed that administration of the formulations containing chelators and MSM (or like permeation enhancers) while effective in treating macromolecular aggregation nevertheless lead to discomfort in patients marked stinging sensation in the eye thus causing patients to discontinue treatment. This may in part be caused by higher concentrations of chelator and/or permeation agent in the formulation.

Artificial tears are used to alleviate eye discomfort by using one or more demulcents including: carboxymethylcellulose, dextran, glycerin, hypromellose, polyethylene glycol 400 (PEG 400), polysorbate, polyvinyl alcohol, povidone, or propylene glycol, among others. The FDA approved the use of artificial tears for treatment of eye discomfort specifying the ingredients and concentrations for such usage. (Food and Drug Administration: “Ophthalmic Drug Products for Over-the-counter Human use; Final Monograph” 21 CFR Parts 349 and 369. Federal Register 1988, 53(43):7076-7093 Available from: www.fda.gov/downloads/Drugs/DevelopmentApprovalProcess/DevelopmentResources/Over-the-CounterOTCDrugs/StatusofOTCRulemakings/ucm094081.pdf). Other ingredients used in artificial tears include emollients which are oily or fat based agents which are used to soften and protect tissues to prevent cracking or drying.

Efforts to solve the problem by using standard concentrations of viscoelastic polymers (such as hydroxypropyl methylcellulose, carboxymethylcellulose, hydroxyethylcellulose and polyvinyl alcohol) as used in artificial tears (˜0.2%). These polymers and concentrations used always produced more stinging, for a longer time, than when the polymers were not used.

### SUMMARY OF THE INVENTION

The present invention is based on the surprising observation that the use of hydroxyethylcellulose (HEC) at significantly increased concentrations (0.5 to 5.0%), there was a significant reduction in stinging.

Without being bound by theory it is postulated that the much higher HEC concentration has such a dramatic reduction in rate of release of the EDTA/MSM, that the stinging is now significantly reduced. The mucus membrane of the eye, especially in corners of the eye near the nose, where most stinging occurs, no longer experiences the levels of chelator/MSM which caused the stinging at lower levels of viscoelastic compounds

In some embodiments, the present invention relates to methods for use of the formulations comprising a transport enhancer (such as MSM) and a chelating agent (such as EDTA) and concentrations of HEC between 0.5% to 5% and an ophthalmologically acceptable inert carrier, for reduction of adverse ophthalmic conditions caused by macromolecular aggregation.

The concentration of HEC can be selected from 0.5%, 0.6%, 0.7%, 0.8%, 0.85% 0.9%, 1.0%, 1.5%, 2.0%, 5.0% or any ranges encompassed by these vales. In particular embodiments HEC is between 0.8% to 1.0%. In one embodiment the concentrations are 1.3% EDTA, 2.7% MSM and 0.85% HEC.

The method involves administering to the subject an effective amount of a formulation composed of a therapeutically effective amount of a chelating agent and an effective transport-enhancing amount of a transport enhancer having the formula (I)

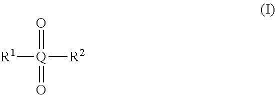

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P.

The transport enhancing agent can be, for example, methylsulfonylmethane (MSM; also referred to as methylsulfone, dimethylsulfone, and DMSO₂), and the chelating agent can be ethylene diamine tetra-acetic acid (EDTA) and the like.

The formulation may be administered in any form suitable for ophthalmic administration including liquid and gel-based compositions. Additionally, in a preferred embodiment, the formulation is entirely composed of components that are naturally occurring and/or as GRAS (“Generally Regarded as Safe”) by the U.S. Food and Drug Administration.

In another embodiment, the present invention provides a method for inhibiting formation of a biofilm on the eye, the method comprising contacting the bacteria with an effective amount of a formulation comprising a transport enhancer (such as MSM), a chelating agent (such as EDTA) and HEC at 0.5% to 5.0%, whereby formation of a biofilm in the eye is inhibited.

A further embodiment of the present invention provides ocular inserts for inhibiting formation of a biofilm, the ophthalmic formulation comprising a formulation comprising a transport enhancer (such as MSM), a chelating agent (such as EDTA) and HEC at 0.5% to 5% and a pharmaceutically acceptable vehicle.

The invention also pertains to a method for the prevention and treatment of adverse ocular conditions, including those that involve oxidative and/or free radical damage in the eye, some of which are also associated with the formation or deposition of macromolecular aggregates. The formulation contains a therapeutically effective amount of an ophthalmologically active agent, a sequestrant of metal cations, e.g., a chelating agent as described above, and a transport enhancer as also described above. These adverse ocular conditions include, by way of example, conditions, diseases, or disorders of the cornea, retina, lens, sclera, and anterior and posterior segments of the eye. An adverse ocular condition as that term is used herein may be a “normal” condition that is frequently seen in aging individuals (e.g., decreased visual acuity and contrast sensitivity) or a pathologic condition that may or may not be associated with the aging process. The latter adverse ocular conditions include a wide variety of ocular disorders and diseases. Aging-related ocular problems that can be prevented and/or treated using the present formulations include, without limitation, opacification (both corneal and lens opacification), cataract formation (including secondary cataract formation) and other problems associated with deposition of lipids, visual acuity impairment, decreased contrast sensitivity, photophobia, glare, dry eye, loss of night vision, narrowing of the pupil, presbyopia, age-related macular degeneration, elevated intraocular pressure, glaucoma, and arcus senilis. By “aging-related” is meant a condition that is generally recognized as occurring far more frequently in older patients, but that may and occasionally do occur in younger people. The formulations can also be used in the treatment of ocular surface growths such as pingueculae and pterygia, which are typically caused by dust, wind, or ultraviolet light, but may also be symptoms of degenerative diseases associated with the aging eye. Another adverse condition that is generally not viewed as aging-related but which can be treated using the present formulation includes keratoconus. It should also be emphasized that the present formulation can be advantageously employed to improve visual acuity, in general, in any mammalian individual. That is, ocular administration of the formulation can improve visual acuity and contrast sensitivity as well as color and depth perception regardless of the patient's age or the presence of any adverse ocular conditions. The formulation is useful for treating adverse ophthalmic conditions in both humans and animals.

In some embodiments, the formulation is effective in alleviating dry eye symptoms, especially dry eyes associated with inflammation.

These and other aspects will become apparent from the following description of the preferred embodiment taken in conjunction with the following drawings, although variations and modifications therein may be affected without departing from the spirit and scope of the novel concepts of the disclosure.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The inventions of these publications, patents and published patent applications referenced in this application are hereby incorporated by reference in their entireties into the present invention. Citation herein of a publication, patent, or published patent application is not an admission the publication, patent, or published patent application is prior art.

As used herein and in the appended claims, the singular forms “a,” “and,” and “the” include plural referents unless the context clearly dictates otherwise. Thus, for example, “a transport enhancer” encompasses a plurality of transport enhancers as well as a single transport enhancer. Reference to “a chelating agent” includes reference to two or more chelating agents as well as a single chelating agent, and so forth. In this specification and in the claims that follow, reference will be made to a number of terms, which shall be defined to have the following meanings:

When referring to a formulation component, it is intended that the term used, e.g., “agent,” encompass not only the specified molecular entity but also its pharmaceutically acceptable analogs, including, but not limited to, salts, esters, amides, prodrugs, conjugates, active metabolites, and other such derivatives, analogs, and related compounds.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause. Unless otherwise indicated herein, either explicitly or by implication, if the term “treatment” (or “treating”) is used without reference to possible prevention, it is intended that prevention be encompassed as well, such that “a method for the treatment of gingivitis” would be interpreted as encompassing “a method for the prevention of gingivitis.”

“Optional” or “optionally present”—as in an “optional substituent” or an “optionally present additive” means that the subsequently described component (e.g., substituent or additive) may or may not be present, so that the description includes instances where the component is present and instances where it is not.

By “pharmaceutically acceptable” is meant a material that is not biologically or otherwise undesirable, e.g., the material may be incorporated into a formulation of the invention without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent. The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition. The term “chelating agent” (or “active agent”) refers to any chemical compound, complex or composition that exhibits a desirable effect in the biological context, i.e., when administered to a subject or introduced into cells or tissues in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “chelating agent” is used, or when a particular chelating agent is specifically identified, it is to be understood that pharmaceutically acceptable salts, esters, amides, prodrugs, active metabolites, isomers, analogs, etc. of the agent are intended as well as the agent per se.

By an “effective” amount or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

The term “controlled release” refers to an agent-containing formulation or fraction thereof in which release of the agent is not immediate, i.e., with a “controlled release” formulation, administration does not result in immediate release of the agent into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein refers to “sustained release” rather than to “delayed release” formulations. The term “sustained release” (synonymous with “extended release”) is used in its conventional sense to refer to a formulation that provides for gradual release of an agent over an extended period of time.

By a “pharmaceutically acceptable” or “ophthalmologically acceptable” component is meant a component that is not biologically or otherwise undesirable, i.e., the component may be incorporated into an ophthalmic formulation of the invention and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration.

The terms “peptide” and “peptidyl” are intended to include any structure comprised of two or more amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids may be replaced by a non-conventional amino acid such as, for example, an isomer or analog of a conventional amino acid (e.g., a D-amino acid), a non-protein amino acid, a post-translationally modified amino acid, an enzymatically modified amino acid, or a construct or structure designed to mimic an amino acid. Peptidyl compounds herein include proteins, oligopeptides, polypeptides, lipoproteins, glycosylated peptides, glycoproteins, and the like.

As will be apparent to those of skill in the art upon reading this invention, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

Unless otherwise indicated, the invention is not limited to specific formulation components, modes of administration, chelating agents, manufacturing processes, or the like, as such may vary.

Unless otherwise defined, all technical and scientific terms used herein have the same meaning as commonly understood by one of ordinary skill in the art to which this invention pertains. In the case of conflict, the present document, including definitions will control.

### Ophthalmic Disorders and Adverse Conditions

All parts of the eye, including the cornea, sclera, trabeculum, iris, lens, vitreous humor, and retina are affected by the aging process, as explained below.

The cornea is the eye's outermost layer. It is the clear, dome-shaped surface that covers the front of the eye. The cornea is composed of five layers. The epithelium is a layer of cells that forms the surface. It is only about 5-6 cell layers thick and quickly regenerates when the cornea is injured. If an injury penetrates more deeply into the cornea, scarring may occur and leave opaque areas, causing the cornea to lose its clarity and luster Immediately below the epithelium is Bowman's membrane, a protective layer that is very tough and difficult to penetrate. The stroma, the thickest layer of the cornea, lies just beneath Bowman's membrane and is composed of tiny collagen fibrils aligned in parallel, an arrangement that provides the cornea with its clarity. Descemet's membrane underlies the stroma and is just above the innermost corneal layer, the endothelium. The endothelium is just one cell layer in thickness, and serves to pump water from the cornea to the aqueous, keeping it clear. If damaged or diseased, these cells will not regenerate.

As the eye ages, the cornea can become more opaque. Opacification can take many forms. The most common form of opacification affects the periphery of the cornea, and is termed “arcus senilis,” or “arcus.” This type of opacification initially involves deposition of lipids into Descemet's membrane. Subsequently, lipids deposit into Bowman's membrane and possibly into the stroma as well. Arcus senilis is usually not visually significant, but is a cosmetically noticeable sign of aging. There are other age related corneal opacifications, however, which may have some visual consequences. These include central cloudy dystrophy of Francois, which affects the middle layers of the stroma, and posterior crocodile shagreen, which is central opacification of the posterior stroma. Opacification, by scattering light, results in progressive reduction of visual contrast and visual acuity.

Opacification of the cornea develops as a result of a number of factors, including, by way of example: degeneration of corneal structure; cross-linking of collagen and other proteins by metalloproteinases; ultraviolet (UV) light damage; oxidation damage; and buildup of substances like calcium salts, protein waste, and excess lipids.

There is no established treatment for slowing or reversing corneal changes other than surgical intervention. For example, opaque structures can be scraped away with a blunt instrument after first removing the epithelium, followed by smoothing and sculpting the corneal surface with a laser beam. In severe cases of corneal scarring and opacification, corneal transplantation has been the only effective approach.

Another common ocular disorder that adversely affects the cornea as well as other structures within the eye is keratoconjunctivitis sicca, commonly referred to as “dry eye syndrome” or “dry eye.” Dry eye can result from a host of causes, and is frequently a problem for older people. The disorder is associated with a scratchy sensation, excessive secretion of mucus, a burning sensation, increased sensitivity to light, and pain. Dry eye is currently treated with “artificial tears,” a commercially available product containing a lubricant such as low molecular weight polyethylene glycol. Surgical treatment, also, is not uncommon, and usually involves insertion of a punctal plug so that lacrimal secretions are retained in the eye. However, both types of treatment are problematic: surgical treatment is invasive and potentially risky, while artificial tear products provide only very temporary and often inadequate relief.

The sclera is the white of the eye. In younger individuals, the sclera has a bluish tinge, but as people grow older, the sclera yellows as a result of age-related changes in the conjunctiva. Over time, UV and dust exposure may result in changes in the conjunctival tissue, leading to pingecula and pterygium formation. These ocular growths can further cause breakdown of scleral and corneal tissue. Currently, surgery, including conjunctival transplantation, is the only accepted treatment for pingeculae and pterygia.

The trabeculum, also referred to as the trabecular meshwork, is a mesh-like structure located at the iris-sclera junction in the anterior chamber of the eye. The trabeculum serves to filter aqueous fluid and control its flow from the anterior chamber into the canal of Schlemm As the eye ages, debris and protein-lipid waste may build up and clog the trabeculum, a problem that results in increased pressure within the eye, which in turn can lead to glaucoma and damage to the retina, optic nerve, and other structures of the eye. Glaucoma drugs can help reduce this pressure, and surgery can create an artificial opening to bypass the trabeculum and reestablish flow of liquid out of the vitreous and aqueous humor. There is, however, no known method for preventing a build-up of debris and protein-lipid waste within the trabeculum.

The Iris and Pupil: With age, dilation and constriction of the iris in response to changes in illumination become slower, and its range of motion decreases. Also, the pupil becomes progressively smaller with age, severely restricting the amount of light entering the eye, especially under low light conditions. The narrowing pupil and the stiffening, slower adaptation, and constriction of the iris over time are largely responsible for the difficulty the aged have in seeing at night and adapting to changes in illumination. The changes in iris shape, stiffness, and adaptability are generally thought to come from fibrosis and cross-linking between structural proteins. Deposits of protein and lipid wastes on the iris over time may also lighten its coloration. Both the light-colored deposits on the iris, and narrowing of the pupil, are very noticeable cosmetic markers of age that may have social implications for individuals. There is no standard treatment for any of these changes, or for changes in iris coloration with age.

With age, the lens yellows, becomes harder, stiffer, and less pliable, and can opacify either diffusely or in specific locations. Thus, the lens passes less light, which reduces visual contrast and acuity. Yellowing also affects color perception. Stiffening of the lens as well as the inability of the muscle to accommodate the lens results in a condition generally known as presbyopia. Presbyopia, almost always occurring after middle age, is the inability of an eye to focus correctly. This age-related ocular pathology manifests itself in a loss of accommodative ability, i.e., the capacity of the eye, through the lens, to focus on near or far objects by changing the shape of the lens to become more spherical (or convex). Both myopic and hyperopic individuals are subject to presbyopia. The age-related loss of accommodative amplitude is progressive, and presbyopia is perhaps the most prevalent of all ocular afflictions, ultimately affecting virtually all individuals during the normal human life span.

These changes in the lens are thought to be due to degenerative changes in the structure of the lens, including glycated crosslinks between collagen fibers, buildup of protein complexes, ultraviolet light degradation of structures, oxidation damage, and deposits of waste proteins, lipids, and calcium salts. Elastic and viscous properties of the lens are dependent on properties of the fiber membranes and cytoskeleton crystallins. The lens fiber membranes are characterized by an extremely high cholesterol to phospholipid ratio. Any changes in these components affect the deformability of the lens membrane. The loss of lens deformability has also been attributed to increased binding of lens proteins to the cell membranes.

Compensatory options to alleviate presbyopia currently include bifocal reading glasses and/or contact lenses, monovision intraocular lenses (IOLs) and/or contact lenses, multifocal IOLs, monovision and anisometropic corneal refractive surgical procedures using radial keratotomy (RK), photorefractive keratomileusis (PRK), and laser-assisted in situ keratomileusis (LASIK). No universally accepted treatments or cures are currently available for presbyopia.

Opacity of the lens results in an abnormal condition generally known as cataract. Cataract is a progressive ocular disease, which subsequently leads to lower vision. Most of this ocular disease is age-related senile cataract. The incidence of cataract formation is thought to be 60-70% in persons in their sixties and nearly 100% in persons eighty years or older. However, at the present time, there is no agent that has been clearly proven to inhibit the development of cataracts. Therefore, the development of an effective therapeutic agent has been desired. Presently, the treatment of cataracts depends upon the correction of vision using eyeglasses, contact lenses, or surgical operations such as insertion of an intra-ocular lens into the capsula lentis after extra-capsular cataract extraction.

In cataract surgery, the incidence of secondary cataract after surgery has been a problem. Secondary cataract is equated with opacity present on the surface of the remaining posterior capsule following extracapsular cataract extraction. The mechanism of secondary cataract is mainly as follows. After excising lens epithelial cells (anterior capsule), secondary cataract results from migration and proliferation of residual lens epithelial cells, which are not completely removed at the time of extraction of the lens cortex, onto the posterior capsule leading to posterior capsule opacification. In cataract surgery, it is impossible to remove lens epithelial cells completely, and consequently it is difficult to always prevent secondary cataract. It is said that the incidence of the above posterior capsule opacification is 40-50% in eyes that do not receive an intracapsular posterior chamber lens implant and 7-20% in eyes which do receive an intracapsular lens implant. Additionally, eye infections categorized as endophthalmitis have also been observed after cataract surgeries.

The Vitreous Humor: Floaters are debris particles that interfere with clear vision by projecting shadows on the retina. There currently is no standard treatment for reducing or eliminating floaters.

A number of changes can occur in the retina with age. Atherosclerotic buildup and leakage in the retinal arteries can lead to macular degeneration as well as reduction of peripheral vision. The rods and cones can become less sensitive over time as they replenish their pigments more slowly. Progressively, all these effects can reduce vision, ultimately leading to partial or complete blindness. Retinal diseases such as age-related macular degeneration have been hard to cure. Current retinal treatments include laser surgery to stop the leaking of blood vessels in the eye.

As alluded to above, current therapeutic attempts to address many ocular disorders and diseases, including aging-related ocular problems, often involve surgical intervention. Surgical procedures are, of course, invasive, and, furthermore, often do not achieve the desired therapeutic goal. Additionally, surgery can be very expensive and may result in significant undesired after-effects. For example, secondary cataracts may develop after cataract surgery and infections may set in. Endophthalmitis has also been observed after cataract surgery. In addition, advanced surgical techniques are not universally available, because they require a very well developed medical infrastructure. Therefore, it would be of significant advantage to provide straightforward and effective pharmacological therapies that obviate the need for surgery.

There have been products proposed to address specific, individual aging-related ocular conditions. For example, artificial tears and herbal formulations have been suggested for treating dry eye syndrome, and other eye drops are available to reduce intraocular pressure, alleviate discomfort, promote healing after injury, reduce inflammation, and prevent infections. However, self-administration of multiple products several times a day is inconvenient, potentially results in poor patient compliance (in turn reducing overall efficacy), and can involve detrimental interaction of formulation components. For example, the common preservative benzalkonium chloride may react with other desirable components such as ethylenediamine tetraacetic acid (EDTA). Accordingly, there is a need in the art for a comprehensive pharmaceutical formulation that can prevent, arrest, and/or reverse a multiplicity of aging-related vision problems and the associated ocular disorders.

Many adverse ocular conditions are associated with the formation, presence, and/or growth of macromolecular aggregates in the eye. Indeed, many pathologies result from or are associated with the deposition and/or aggregation of proteins, other peptidyl species, lipoproteins, lipids, polynucleotides, and other macromolecules throughout the body. For example, Advanced Glycation Endproducts (also termed AGEs) are formed by the binding of glucose or other reducing sugars to proteins, lipoproteins and DNA by a process known as non-enzymatic glycation, followed by cross-linking. These cross-linked macromolecules stiffen connective tissue and lead to tissue damage in the kidney, retina, vascular wall and nerves. AGEs have, in fact, been implicated in the pathogenesis of a variety of debilitating diseases such as diabetes, atherosclerosis, Alzheimer's and rheumatoid arthritis, as well as in the normal aging process. Peptidyl deposits are also associated with Alzheimer's disease, sickle cell anemia, multiple myeloma, and prion diseases. Lipids, particularly sterols and sterol esters, represent an additional class of biomolecules that form pathogenic deposits in vivo, including atherosclerotic plaque, gallstones, and the like. To date, there has been no single formulation identified capable of treating a plurality of such disorders.

### Chelating Agent/Chelator

Chelation is a chemical combination with a metal in complexes in which the metal is part of a ring. An organic ligand is called a chelator or chelating agent, the chelate is a metal complex. The larger number of ring closures to a metal atom the more stable is the compound. The stability of a chelate is also related to the number of atoms in the chelate ring. Monodentate ligands which have one coordinating atom like H₂O or NH₃ are easily broken apart by other chemical processes, whereas polydentate chelators, donating multiple binds to metal ion, provide more stable complexes. Chlorophyll, a green plant pigment, is a chelate that consists of a central magnesium atom joined with four complex chelating agent (pyrrole ring). Heme is an iron chelate which contains iron (II) ion in the center of the porphyrin. Chelating agents offers a wide range of sequestrants to control metal ions in aqueous systems. By forming stable water soluble complexes with multivalent metal ions, chelating agents prevent undesired interaction by blocking normal reactivity of metal ions. EDTA (ethylenediamine tetraacetate) is a good example of common chelating agents which have nitrogen atoms and short chain carboxylic groups.

Examples of chelators of iron and calcium include, but are not limited to, Diethylene triamine pentaacetic acid (DTPA), ethylene diamine tetraacetic acid (EDTA), nitrilotriacetic acid (NTA), 1,3-propylene diamine tetraacetic acid (PDTA), Ethylene diamine disuccinic acid (EDDS), and ethylene glycol tetraacetic acid (EGTA). Any suitable chelating agent known in the art, which is biologically safe and able to chelate iron, calcium or other metals, is suitable for the invention.

Compounds useful as chelating agents herein include any compounds that coordinate to or form complexes with a divalent or polyvalent metal cation, thus serving as a sequestrant of such cations. Accordingly, the term “chelating agent” herein includes not only divalent and polyvalent ligands (which are typically referred to as “chelators”) but also monovalent ligands capable of coordinating to or forming complexes with the metal cation.

Suitable biocompatible chelating agents useful in conjunction with the present invention include, without limitation, monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing. Other exemplary chelating agents include: phosphates, e.g., pyrophosphates, tripolyphosphates, and hexametaphosphates.

EDTA and ophthalmologically acceptable EDTA salts are particularly preferred, wherein representative ophthalmologically acceptable EDTA salts are typically selected from diammonium EDTA, disodium EDTA, dipotassium EDTA, triammonium EDTA, trisodium EDTA, tripotassium EDTA, and calcium disodium EDTA.

EDTA has been widely used as an agent for chelating metals in biological tissue and blood, and has been suggested for inclusion in various formulations. For example, U.S. Pat. No. 6,348,508 to Denick Jr. et al. describes EDTA as a sequestering agent to bind metal ions. In addition to its use as a chelating agent, EDTA has also been widely used as a preservative in place of benzalkonium chloride, as described, for example, in U.S. Pat. No. 6,211,238 to Castillo et al. U.S. Pat. No. 6,265,444 to Bowman et al. discloses use of EDTA as a preservative and stabilizer. However, EDTA has generally not been applied topically in any significant concentration formulations because of its poor penetration across biological membranes and biofilms including skin, cell membranes and even biofilms like dental plaque.

Among the chelating/sequetering materials which may be included in the compositions there may be mentioned biocompatible chelating agents include, without limitation, monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing.

Other exemplary chelating agents include: phosphates, e.g., pyrophosphates, tripolyphosphates, and hexametaphosphates. Other exemplary chelating agents include: phosphates, e.g., pyrophosphates, tripolyphosphates, and hexametaphosphates; chelating antibiotics such as chloroquine and tetracycline; nitrogen-containing chelating agents containing two or more chelating nitrogen atoms within an imino group or in an aromatic ring (e.g., diimines, 2,2′-bipyridines, etc.); and polyamines such as cyclam (1,4,7,11-tetraazacyclotetradecane), N—(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbamoyl) propanoylamino]pentyl]-N-hydroxy-butane diamide); also known as desferrioxamine B, desferoxamine B, DFO-B, DFOA, DFB or desferal), deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

Additional, suitable biocompatible chelating agents which may be useful for the practice of the current disclosure include EDTA-4-aminoquinoline conjugates such as ([2-(Bis-ethoxycarbonylmethyl-amino)-ethyl]-{[2-(7-chloro-quinolin-4-ylamino)-ethylcarbamoyl]-methyl}-amino)-acetic acid ethyl ester, ([2-(Bis-ethoxycarbonylmethyl-amino)-propyl]-{[2-(7-chloro-quinolin-4-ylamino)-ethylcarbamoyl]-methyl}-amino)-acetic acid ethyl ester, ([3-(Bis-ethoxycarbonylmethyl-amino)-propyl]-{[2-(7-chloro-quinolin-4-ylamino)-ethylcarbamoyl]-methyl}-amino)-acetic acid ethyl ester, ([4-(Bis-ethoxycarbonylmethyl-amino)-butyl]-{[2-(7-chloro-quinolin-4-ylamino)-ethylcarbamoyl]-methyl}-amino)-acetic acid ethyl ester, ([2-(Bis-ethoxymethyl-amino)-ethyl]-{[2-(7-chloro-quinolin-4-ylamino)-ethylcarbamoyl]-methyl}-amino)-acetic acid ethyl ester, ([2-(Bis-ethoxymethyl-amino)-propyl]-{[2-(7-chloro-quinolin-4-ylamino)-ethylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]-{[2-(7-chloro-quinolin-4-ylamino)-ethylcarbamoyl]-methyl}-amino)-acetic acid ethyl ester as described in Solomon et al., Med. Chem. 2: 133-138, 2006.

Additionally, natural chelators including, but not limited to citric acid, phytic acid, lactic acid, acetic acid and their salts. Other natural chelators like but not limited to curcumin (turmeric).

In some embodiments, the chelating agent incorporated in the formulation is a prochelator. A prochelator is any molecule that is converted to a chelator when exposed to the appropriate chemical or physical conditions. For example, BSIH (isonicotinic acid [2-(4,4,5,5-tetramethyl-[1,3,2]dioxaborolan-2-yl)-benzylidene]-hydrazide) prochelators are converted by hydrogen peroxide into SIH (salicylaldehyde isonicotinoyl hydrazone) iron-chelating agents that inhibit iron-catalyzed hydroxyl radical generation.

The inactivated metal ion sequestering agent is sometimes referred to herein as a “prochelator,” although sequestration of metal ions can involve sequestration and complexation processes beyond the scope of chelation per se. The term “prochelator” is analogous to the term “prodrug” insofar as a prodrug is a therapeutically inactive agent until activated in vivo, and the prochelator, as well, is incapable of sequestering metal ions until activated in vivo.

Transport Enhancer: The transport enhancer is selected to facilitate the transport of a chelating agent through the tissues, extra-cellular matrices, and/or cell membranes of a body. An “effective amount” of the transport enhancer represents an amount and concentration within a formulation of the invention that is sufficient to provide a measurable increase in the penetration of a chelating agent through one or more of the sites in a subject than would otherwise be the case without the inclusion of the transport enhancer within the formulation.

In certain instances, the transport enhancer may be present in a formulation of the invention in an amount that ranges from about 0.01 wt. % or less to about 30 wt. % or more, typically in the range of about 0.1 wt. % to about 20 wt. %, more typically in the range of about 1 wt. % to about 11 wt. %, and most typically in the range of about 2 wt. % to about 8 wt. %, for instance, 5 wt. %.

The transport enhancer is generally of the formula (I)

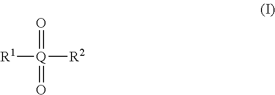

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P. Compounds wherein Q is S and R¹ and R² are C₁-C₃ alkyl are preferred, with methylsulfonylmethane (MSM) being the optimal transport enhancer.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups are contain one aromatic ring or two fused or linked aromatic rings, e.g., phenyl, naphthyl, biphenyl, diphenylether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl, and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

In one embodiment, then, a method is provided for eliminating or reducing the size of an aggregate of macromolecules in the eye. The method involves administering to the eye(s) of a patient a therapeutically effective amount of a sterile ophthalmic formulation comprised of (a) a noncytotoxic chelating agent containing at least three negatively charged chelating atoms, (b) a charge-masking agent containing at least one polar group and (c) HEC at a concentration greater than 0.5%, and inert ophthalmically acceptable carriers. The polar group of the charge-masking agent contains at least one and preferably at least two heteroatoms having a Pauling electronegativity greater than about 3.00, wherein the heteroatoms are preferably oxygen atoms. The molar ratio of the charge-masking agent to the chelating agent is sufficient to ensure that substantially all negatively charged chelating atoms are associated with at least one of the aforementioned heteroatoms on the charge-masking agent. The formulation may be applied to the eye in any form suitable for ocular drug administration, e.g., as a solution or suspension for administration as eye drops or eye washes, as an ointment, or in an ocular insert that can be implanted in the conjunctiva, sclera, pars plana, anterior segment, or posterior segment of the eye. Such inserts provide for controlled release of the formulation to the ocular surface, typically sustained release over an extended time period.

The formulation may also be applied to the skin around the eye for penetration therethrough, insofar as the compound used as the charge-masking agent, e.g., methylsulfonylmethane, also serves as a penetration enhancer allowing permeation of the formulation through the skin.

In another embodiment, the formulation is effective in alleviating dry eye symptoms, especially dry eyes associated with inflammation and can be used in methods to treat dry eyes. Subjects with extreme dry eyes, such as those with Sjogren's Syndrome, may yet experience some stinging sensation due to the extreme dryness of their eyes.

Accordingly, the chelating agent is multifunctional in the context of the present invention, insofar as the agent serves to decrease unwanted proteins or peptides, prevent formation of mineral deposits, and/or reduce mineral deposits that have already formed, and reduce calcification, in addition to acting as a preservative and stabilizing agent.

The formulation also includes an effective amount of a transport enhancer that facilitates penetration of the formulation components through cell membranes, tissues, and extracellular matrices. The “effective amount” of the transport enhancer represents a concentration that is sufficient to provide a measurable increase in penetration of one or more of the formulation components through membranes, tissues, and extracellular matrices as just described. Suitable transport enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred.

MSM is an odorless, highly water-soluble (34% w/v @ 79° F.) white crystalline compound with a melting point of 108-110° C. and a molecular weight of 94.1 g/mol. MSM serves as a multifunctional agent herein, insofar as the agent not only increases cell membrane permeability, but also acts as a “transport facilitating agent” (TFA) that aids in the transport of one or more formulation components to the eye. Furthermore, MSM per se provides medicative effects, and can serve as an anti-inflammatory agent as well as an analgesic. MSM also acts to improve oxidative metabolism in biological tissues, and is a source of organic sulfur, which assists in the reduction of scarring. MSM additionally possesses unique and beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for formation of van der Waal associations, i.e., between the methyl groups and nonpolar (e.g., hydrocarbyl) segments of other molecules. Ideally, the concentration of MSM in the present formulations is in the range of about 0.1 wt. % to 40 wt. %, or from about 1 wt. % to about 4, 5, 6, 7, 8, 10, 15 wt. %, and preferably between about 1.5 wt. % to 8.0 wt. %.

Other optional additives in the present formulations include secondary enhancers, i.e., one or more additional transport enhancers. For example, formulation of the invention can contain added DMSO. Since MSM is a metabolite of DMSO (i.e., DMSO is enzymatically converted to MSM), incorporating DMSO into an MSM-containing formulation of the invention will tend to gradually increase the fraction of MSM in the formulation. DMSO also serves as a free radical scavenger, thereby reducing the potential for oxidative damage. If DMSO is added as a secondary enhancer, the amount is preferably in the range of about 1.0 wt. % to 2.0 wt. % of the formulation, and the weight ratio of MSM to DMSO is typically in the range of about 1:50 to about 50:1.

Hydroxyethyl Cellulose (also known and available as HEC, HESPAN; TYLOSE P; NATROSOL; HETASTARCH) is a non-ionic cellulose ether made through a series of chemical processes, with the natural polymer celluloses as raw materials. It is odorless, tasteless, and non-toxic in the shape of white to off-white powders or granules. It can be dissolved in water to form a transparent viscous solution. The solubility of HEC in water is: ≤5 wt. % at 20° C. The CAS DataBase Reference for HEC is 9004-62-0.

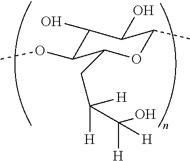

Hydroxyethyl cellulose is soluble in hot or cold water and does not precipitate by heat or boiling which enables it to have a wide range of solubility and viscosity characteristics, as well as non-thermal gelation. It is non-ionic and can coexist with a wide range of other water-soluble polymers, surfactants, and salts, and is a fine colloidal thickener for the solution containing a high concentration of electrolytes. Hydroxyethyl cellulose can be dispersed in cold water without agglomeration, but dissolution rate is slower, and generally it requires about 30 minutes. With heat or adjusting the pH value to 8-10, it can be rapidly dissolved.

The formulation can also include a microcirculatory enhancer, i.e., an agent that serves to enhance blood flow within the capillaries. The microcirculatory enhancer can be a phosphodiesterase (PDE) inhibitor, for instance a Type (I) PDE inhibitors. Such compounds, as will be appreciated by those of ordinary skill in the art, act to elevate intracellular levels of cyclic AMP (cAMP). A preferred microcirculatory enhancer is vinpocetine, also referred to as ethyl apovincamin-22-oate. Vinpocetine, a synthetic derivative of vincamine, a Vinca alkaloid, is particularly preferred herein because of its antioxidant properties and protection against excess calcium accumulation in cells. Vincamine is also useful as a microcirculatory enhancer herein, as are Vinca alkaloids other than vinpocetine. Preferably, any microcirculatory enhancer present, e.g., vinpocetine, represents about 0.01 wt. % to about 0.2 wt. %, preferably in the range of about 0.02 wt. % to about 0.1 wt. % of the formulation.

### Formulations

A variety of means can be used to formulate the compositions of the invention. Techniques for formulation and administration may be found in “Remington: The Science and Practice of Pharmacy,” Twentieth Edition, Lippincott Williams & Wilkins, Philadelphia, Pa. (1995). For human or animal administration, preparations should meet sterility, pyrogenicity, general safety and purity standards comparable to those required by the FDA. Administration of the pharmaceutical formulation can be performed in a variety of ways, as described herein.

Other possible additives for incorporation into the formulations that are at least partially aqueous include, without limitation, thickeners, isotonic agents, buffering agents, and preservatives, providing that any such excipients do not interact in an adverse manner with any of the formulation's other components. It should also be noted that preservatives are not generally necessarily in light of the fact that the selected chelating agent itself serves as a preservative. Suitable thickeners will be known to those of ordinary skill in the art of formulation, and include, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers).

The preferred amount of any thickener is such that a viscosity above 10,000 cps is provided, as a gel having a viscosity above this figure generally considered optimal for both comfort and retention of the formulation on the eye. Any suitable isotonic agents and buffering agents commonly used in ophthalmic formulations may be used, providing the pH of the formulation is maintained in the range of about 4.5 to about 9.0, preferably in the range of about 6.8 to about 7.8, and optimally at a pH of about 7.4. Preferred buffering agents include carbonates such as sodium and potassium bicarbonate.

However, an effective thickening agent must be used in an amount that also exhibits the key properties of enabling uses of lower concentrations of chelator/MSM combination to achieve significant effect without causing symptoms of discomfort in the eye, such as severe stinging.

The formulations of the invention also include a pharmaceutically acceptable ophthalmic carrier or vehicle, which will depend on the particular type of formulation. For example, the formulations of the invention can be provided as an ophthalmic solution or suspension, in which case the carrier is at least partially aqueous. Ideally, ophthalmic solutions, which may be administered as eye drops, are aqueous solutions. The formulations may also be ointments, in which case the pharmaceutically acceptable carrier is composed of an ointment base. Preferred ointment bases herein have a melting or softening point close to body temperature, and any ointment bases commonly used in ophthalmic preparations may be advantageously employed. Common ointment bases include petrolatum and mixtures of petrolatum and mineral oil. Suitable pharmaceutical formulations and dosage forms may be prepared using conventional methods known to those in the field of pharmaceutical formulation and described in the pertinent texts and literature, e.g., in Remington: The Science and Practice of Pharmacy, cited previously herein.

The formulations of the invention may also be prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application into the eye. Such gels are liquid at room temperature but gel at higher temperatures (and thus termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® trade name from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5μ to about 10μ.

The formulations may also be incorporated into a sterile ocular insert that provides for controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art, and are typically composed of a water-swellable, gel-forming polymer such as collagen, polyvinyl alcohol, or a cellulosic polymer. Another type of insert that can be used to deliver the present formulation is a diffusional implant in which the formulation is contained in a central reservoir enclosed within a permeable polymer membrane that allows for gradual diffusion of the formulation out of the implant. Osmotic inserts may also be used, i.e., implants in which the formulation is released as a result of an increase in osmotic pressure within the implant following application to the eye and subsequent absorption of lachrymal fluid.

The chelating agent may be administered, if desired, in the form of a salt, ester, crystalline form, hydrate, or the like, provided it is pharmaceutically acceptable. Salts, esters, etc. may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

The amount of chelating agent administered will depend on a number of factors and will vary from subject to subject and depend on the particular chelating agent, the particular disorder or condition being treated, the severity of the symptoms, the subject's age, weight and general condition, and the judgment of the prescribing physician. The term “dosage form” denotes any form of a pharmaceutical composition that contains an amount of chelating agent and transport enhancer sufficient to achieve a therapeutic effect with a single administration or multiple administrations. The frequency of administration that will provide the most effective results in an efficient manner without overdosing will vary with the characteristics of the particular active agent, including both its pharmacological characteristics and its physical characteristics, such as hydrophilicity.

In a further embodiment, the formulation can include an additional ophthalmologically active agent, such as may be selected from, for instance: anti-infective or antibiotic agents including fluoroquinolones such as ciprofloxacin, levofloxacin, gentafloxacin, ofloxacine, tetracycline, chlortetracycline, bacitracin, neomycin, polymyxin, gramicidin, oxytetracycline, chloramphenicol, gentamycin, and erythromycin; anti-inflammatory agents such as hydrocortisone, dexamethasone, fluocinolone, prednisone, prednisolone, methylprednisolone, fluorometholone, betamethasone and triamcinolone; anti-angiogenesis drugs including thalidomide, VEGF inhibitors, and matrix metaloproteinaise (MMP) inhibitors; anti-neoplastic agents; and dry-eye medicaments such as cyclosporine and mitomycin. Additional examples of ophthalmologically active agents that may be incorporated into the present formulations include anesthetics, analgesics, cell transport/mobility impeding agents; anti-glaucoma drugs including beta-blockers such as timolol, betaxolol, atenolol, etc; carbonic anhydrase inhibitors such as acetazolamide, methazolamide, dichlorphenamide, and diamox; neuroprotectants such as nimodipine and related compounds; antibacterials such as sulfonamides, sulfacetamide, sulfamethizole and sulfisoxazole; anti-fungal agents such as fluconazole, nitrofurazone, amphotericine B, ketoconazole, and related compounds; anti-viral agents such as trifluorothymidine, acyclovir, ganciclovir, dideoxyinosine (DDI), zidovudine (AZT), foscamet, vidarabine, trifluorouridine, idoxuridine, and ribavirin; protease inhibitors and anti-cytomegalovirus agents; antiallergenics such as methapyriline, chlorpheniramine, pyrilamine and prophenpyridamine; and decongestants such as phenylephrine, naphazoline, and tetrahydrazoline.

Typical ophthalmologically active agents that can be incorporated into the present formulations include, without limitation, aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, a pharmaceutically acceptable salt thereof, or a combination of any of the foregoing.

The formulations of the invention may also be prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application to the eye. Such gels are liquid at room temperature but gel at higher temperatures (and thus termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® tradename from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5 μm to about 10 μm.

Methods of using the formulation of the invention are also contemplated. The formulations of the invention are useful in treating a wide variety of conditions associated with the formation and/or deposition of macromolecular aggregates. Numerous medical pathologies are caused or exacerbated by the in vivo formation or deposition of macromolecular aggregates, including crystalline aggregates, fibrillar aggregates, and amorphous aggregates. Certain peptidyl compounds, including selected oligopeptides, polypeptides, and proteins, are known to form crystals and fibrils that are associated with various medical conditions, disorders, and diseases. For example, amyloid peptides, particularly .beta.-amyloid, are known to form ordered fibrillar aggregates that comprise the extracellular and cerebrovascular senile plaques associated with Alzheimer's disease. See Han et al. (1995), “The Core Alzheimer's Peptide NAC Forms Amyloid Fibrils which Seed and are Seeded by .beta.-Amyloid: is NAC a Common Trigger or Target in Neurodegenerative Disease?” Chemistry and Biology 2:163-169; Serpell et al. (2000), “Molecular Structure of a Fibrillar Alzheimer's A.beta.,” Biochemistry 39:13269-13275; Jarrett and Lansbury (1992), “Amyloid Fibril Formation Requires a Chemically Discriminating Nucleation Event: Studies of an Amyloidogenic Sequence from the Bacterial Protein OsmB,” Biochemistry 31(49):12345-12352; and Jarrett et al. (1993), “The Carboxy Terminus of the Beta Amyloid Protein is Critical for the Seeding of Amyloid Formation: Implications for the Pathogenesis of Alzheimer's Disease,” Biochemistry 32:4693-4697. The prion diseases, e.g., the class of diseases known as the transmissible spongiform encephalopathies, are also characterized by abnormal protein deposition in brain tissue, in which the deposits are comprised of fibrillar amyloid plaques formed primarily from the prion protein (PrP). Such diseases include scrapie transmissible mink encephalopathy, chronic wasting disease of mule deer and elk, feline spongiform encephalopathy, and bovine spongiform encephalopathy (“mad cow disease”) in animals, and Kuru, Creutzfeldt-Jakob disease, Gerstmann-Struessler-Scheinker disease, and fatal familial insomnia in humans. It has been proposed that a 15-mer amino acid sequence, PrP96-111, is responsible for initiating prion formation in vivo by providing a seed for amyloid fiber formation. See Come et al. (1993), “A Kinetic Model for Amyloid Formation in the Prion Diseases: Importance of Seeding,” Proc Natl Acad Sc. USA 90:5959-5963. Fibrillin, associated with Martan's disease, is another example of a protein that forms an ordered fibrillar structure that causes an adverse medical condition. Fibrillar plaques formed from various collagens are also associated with certain medical pathologies, e.g., cardiac diseases and collagenofibrotic glomerulopathy; see Rossi et al. (2001), “Connective Tissue Skeleton in the Normal Left Ventricle and in Hypertensive Left Ventricle Hypertrophy and Chronic Chagasic Monocarditis,” Med Sci Mon 7:820-832; Yasuda et al. (1999), “Collagenofibrotic Glomerulopathy: A Systemic Disease,” Am J Kidney Dis 33:123-127.

Other similarly problematic biomolecules can be treated, such as cystine, which forms crystal deposits in bone marrow (associated with rickets and synovitis), the renal tubule and gastrointestinal tract (associated with cystinuria), and a variety of other body tissues, including the kidneys, eyes, and thyroid glands (associated with cystinosis, including the severe form of the disease, nephropathic cystinosis, or Fanconi's syndrome).

In some compositions, the proportion of the EDTA to MSM is in the range of about 1:100-100:1, and the percentages of EDTA and MSM in the composition are in the ranges of about 0.1% to 15% and about 0.1% to 40% by weight, respectively.

The formulation also contains an effective thickening agent used in an amount that also exhibits the key properties of enabling uses of lower concentrations of chelator/MSM combination to achieve significant effect without causing symptoms of discomfort in the eye, such as severe stinging. Swellable viscoelastic cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC) are preferred. HEC at concentrations of 0.5% to 1.5% and specifically at 0.8% to 1.0% is preferred.

One major improvement of the formulations disclosed herein over previously tested ophthalmic formulations is a significant reduction in discomfort associated with use of prior formulations. Attempts to alleviate such discomforts, such as stinging by using the standard concentrations (˜0.2%) of viscoelastic polymers (such as hydroxypropyl methylcellulose, carboxymethylcellulose, hydroxyethylcellulose and polyvinyl alcohol) as used in artificial tears were unsuccessful. These polymers and concentrations used always produced more stinging, for a longer time, than when the polymers were not used. With HEC, at lower concentrations (e.g., 0.3%), stinging occurred.

Surprisingly, when the concentration of HEC was significantly increased to 0.8 to 1%, there was a significant reduction in stinging. effectively, in the presence of such higher concentrations of HEC, the mucus membrane of the eye, especially in corners pf the eye near the nose, where most stinging occurs, was not experiencing the levels of EDTA/MSM which caused the stinging at lower levels of viscoelastic compounds.

At previously used concentrations of 2.6% EDTA with 5.4% MSM there was intense discomfort manifested as a severe stinging sensation upon initial application. The stinging sensation did dissipate over time suggesting that as lower concentrations of the gradually dissipating chelator/MSM combination, the stinging sensation did not occur.

A second surprising effect was a significant reduction of chelator/MSM levels required for effectiveness. With the slower release of EDTA/MSM from the viscous HEC solution, the amount of EDTA/MSM making its way into the aqueous and vitreous of the eye is greatly increased. Only half of the concentration of EDTA/MSM was required in the presence of higher HEC concentrations in order to get the same clinical effect as without HEC. 1.3% EDTA, 2.7% MSM and 0.8% HEC is as effective as 2.6% EDTA with 5.4% MSM.

### EXAMPLES

The following examples are put forth so as to provide those skilled in the art with a complete invention and description of how to make and use embodiments in accordance with the invention, and are not intended to limit the scope of what the inventors regard as their discovery. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1: Increase in Residence Time of MSM/EDTA Solutions with Increasing Concentration of HEC

The MSM/EDTA eye drops were made with varying concentrations of HEC, and tested in human eyes to estimate their residence times. In eye drops made without HEC, subjects can feel the presence of the EDTA in the nasal passage within seconds of applying the eye drops. The time interval between application of the eye drop and the perception of the presence of the EDTA were taken to be equivalent to the residence time in the eye. The results for five concentrations of HEC in the eye drops are shown in the table below.

| | Mean time in seconds of | |
|---|---|---|
| Concentration of HEC | perception in Nasal passage | |
| 0% | 4.6 | seconds |
| 0.25% | 25.1 | seconds |
| 0.5% | 40.3 | seconds |
| 0.75% | 77.6 | seconds |
| 1% | 133.1 | seconds |

At low concentrations, because of the stinging sensation, the subject is aware that the drop is present. It stings more with low concentrations. At higher (0.75 and 1%) the stinging sensation is markedly reduced.

This demonstrated that increasing the viscosity of the eye drops increases the time taken by the eye drops to travel through the lacrimal puncta into the nasal passage in a non-linear fashion. The actual residence time in the eye may be lower than these numbers.

### Example 2: Enhanced Permeation of Porcine Intestinal Membrane by Ferric Sodium EDTA with Use of Hydroxyethyl Cellulose in Aqueous Medium

The following experimental solutions were prepared. Control: 1% ferric sodium EDTA prepared in distilled water. Test Solutions: 1% ferric sodium EDTA prepared in 1%, 3%, and 5% hydroxyethyl cellulose (HEC) with distilled water

| Solutions Tested | Time (in sec) | Absorbance (at 255 nm) |
|---|---|---|
| 0% HEC (Control | 5 | 0.120 |
| 1% HEC | 30 | 0.258 |
| 3% HEC | 60 | 0.266 |
| 5% HEC | 120 | 0.271 |

### Example 3: Exemplary Eye Drop Formulation According to the Invention

Formulation A was prepared as follows: High purity de-ionized (DI) water (500 ml) was filtered via a 0.2 micrometer filter. MSM, EDTA, and HEC were added to the filtered DI water, and mixed until visual transparency was achieved, indicating dissolution. The mixture was poured into 10 mL bottles each having a dropper cap. On a weight percent basis, the eye drops had the following composition: MSM 2.7% w/w; di-sodium EDTA 1.3% w/w; HEC 0.85% w/w.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claim.

What is claimed is:

1. An ophthalmic formulation for treating adverse ophthalmic conditions, comprising:
(a) a chelating agent or salts thereof;
(b) a transport enhancer which is a charge masking agent;
(c) a concentration of a thickener which is a viscoelastic material in an amount sufficient to reduce side-effects and increase effectiveness; and
(d) a pharmaceutically acceptable inactive vehicle;
wherein the chelating agent and the transport enhancer are present in a proportion effective to bring about a significant reduction in macromolecular aggregation in the eye to which it is applied, and
wherein the percentage of chelator is about 0.1% to 15% and the percentage of transport in the composition is about 0.1% to 40% by weight, respectively.

2. The formulation of claim 1, wherein the transport enhancer is MSM.

3. The formulation of claim 1, wherein the amount of MSM is less than 5%.

4. The formulation of claim 2, wherein the proportion of the chelator to MSM is in the range of about 10:1-1:20.

5. The formulation of claim 1, wherein the viscoelastic polymer is selected from hydroxypropyl methylcellulose, carboxymethylcellulose, hydroxyethylcellulose (HEC) and polyvinyl alcohol.

6. The formulation of claim 1, wherein the viscoelastic polymer is hydroxyethylcellulose (HEC).

7. The formulation of claim 6, wherein the concentration of HEC is between 0.5% and 5.0%.

8. The formulation of claim 6, wherein the concentration of HEC is between 0.5% and 1.0%.

9. The formulation of claim 6, wherein the concentration of HEC is between 0.8% and 0.85%.

10. The formulation of claim 1, wherein the chelating agent is selected from ethylenediamine tetraacetic acid (EDTA), ethylene glycol tetraacetic acid (EGTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ArPA), citric acid, acetic acid and acceptable salts thereof, and any combinations thereof.

11. The formulation of claim 10, wherein the EDTA salt is selected from diammonium EDTA, disodium EDTA, dipotassium EDTA, triammonium EDTA, trisodium EDTA, tripotassium EDTA, tetrasodium EDTA, tetrapotassium EDTA, calcium disodium EDTA, and combinations thereof.

12. The formulation of claim 1, wherein the chelating agent is selected from phosphates, pyrophosphates, tripolyphosphates, and hexametaphosphates.

13. The formulation of claim 1, wherein the chelating agent is a chelating antibiotic, chloroquine or tetracycline.

14. The formulation of claim 1, wherein the chelating agent is a nitrogen-containing chelating agents containing two or more chelating nitrogen atoms within an imino group or in an aromatic ring, diimines, or 2,2′-bipyridines.

15. The formulation of claim 1, wherein the chelating agent is a polyamine selected from cyclam (1,4,7,11-tetraazacyclotetradecane), N—(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbamoyl) propanoylamino]pentyl]-N-hydroxy-butane diamide), desferrioxamine B, desferoxamine B, DFO-B, DFOA, DFB, desferal, deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

16. The formulation of claim 1, wherein the chelating agent is a EDTA-4-aminoquinoline conjugate selected from ([2-(Bis-ethoxycarbonylmethyl-amino)-ethyl]-{[2-(7-chloro-quinolin-4-ylamino)-ethylcarbamoyl]-methyl}-amino)-acetic acid ethyl ester, ([2-(Bis-ethoxycarbonylmethyl-amino)-propyl]-{[2-(7-chloro-quinolin-4-ylamino)-ethylcarbamoyl]-methyl}-amino)-acetic acid ethyl ester, ([3-(Bis-ethoxycarbonylmethyl-amino)-propyl]-{[2-(7-chloro-quinolin-4-ylamino)-ethylcarbamoyl]-methyl}-amino)-acetic acid ethyl ester, ([4-(Bis-ethoxycarbonylmethyl-amino)-butyl]-{[2-(7-chloro-quinolin-4-ylamino)-ethylcarbamoyl]-methyl}-amino)-acetic acid ethyl ester, ([2-(Bis-ethoxymethyl-amino)-ethyl]-{[2-(7-chloro-quinolin-4-ylamino)-ethylcarbamoyl]-methyl}-amino)-acetic acid ethyl ester, ([2-(Bis-ethoxymethyl-amino)-propyl]-{[2-(7-chloro-quinolin-4-ylamino)-ethylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]-{[2-(7-chloro-quinolin-4-ylamino)-ethylcarbamoyl]-methyl}-amino)-acetic acid ethyl ester.

17. The formulation of claim 1, wherein the chelating agent is a natural chelator selected from citric acid, phytic acid, lactic acid, acetic acid and their salts and curcumin.

18. The formulation of claim 1, wherein the vehicle is aqueous.

19. The formulation of claim 1, wherein administration of the formulation reduces macromolecular aggregates in the eye.

20. The formulation of claim 19, wherein the macromolecular aggregates are peptidyl compounds.

21. The formulation of claim 19, wherein the macromolecular aggregates are proteins.

22. The formulation of claim 19, wherein the macromolecular aggregates are lipoproteins.

23. The formulation of claim 1, wherein the formulation comprises an ocular insert.

24. The formulation of claim 1, wherein the formulation is for timed release.

25. The formulation of claim 19, wherein the macromolecular aggregates are

26. The formulation of claim 1, wherein the formulation comprises: MSM at 2.7% w/w; di-sodium EDTA at 1.3% w/w; and HEC 0.85% w/w.

27. A method for alleviating an indication of adverse ocular syndrome by administering to the eye of a subject in need thereof, the formulation of claim 1.

28. The method of claim 27, wherein the adverse ocular condition is accumulation of an aggregate of macromolecules in the eye.

29. The method of claim 27, wherein the formulation comprises amounts of the chelator and permeation enhancer that are at least 75% as effective in the presence of the viscoelastic polymer as compared to the effectiveness in the absence of the viscoelastic polymer.

30. The method of claim 27, wherein the formulation comprises MSM as a permeation enhancer MSM and HEC as a viscoelastic polymer.

31. The method of claim 30, wherein the formulation used comprises: MSM at 2.7% w/w; di-sodium EDTA at 1.3% w/w; and HEC 0.85% w/w.

32. The method of claim 27, wherein the formulation comprises amounts of the chelator, permeation enhancer and viscoelastic polymer produces significantly reduced stinging sensation in the eye of the subjecting the presence of the viscoelastic polymer as compared to the stinging sensation in the absence of the viscoelastic polymer.

33. The method of claim 27, wherein the formulation comprises MSM as a permeation enhancer MSM and HEC as a viscoelastic polymer.

34. The method of claim 30, wherein the formulation used comprises: MSM at 2.7% w/w; di-sodium EDTA at 1.3% w/w; and HEC 0.85% w/w.

35. A formulation for treating adverse ophthalmic conditions while reducing stinging sensations in the eye, the formulation comprising:
(a) a chelating agent or salts thereof;
(b) a charge masking agent that is methylsulfonylmethane (MSM);
(c) a thickener which is hydroxyethylcellulose (HEC) in a concentration between 0.5% to 5.0%; and
(d) a pharmaceutically acceptable inactive vehicle;
wherein the concentration of HEC is sufficient to reduce release of the chelating agent/MSM combination in the eye to maintain a concentration level below that at which a stinging sensation occurs,
wherein the concentration of HEC is sufficient to retain the chelating agent/MSM in the eye for a period effective to bring about a significant reduction in the adverse ophthalmic condition, and
wherein the percentage of chelator is about 0.1% to 3.0% and the percentage of transport in the composition is about 0.1% to 6.0% by weight, respectively.

36. The formulation of claim 35, wherein the adverse ophthalmic condition is caused by macromolecular aggregation.

37. The formulation of claim 35, wherein the adverse ophthalmic condition is caused by dry eye syndrome.

38. The formulation of claim 37, wherein the dry eye syndrome is caused by inflammation.
